(19) United States
(12) Patent Application Publication
(10) Pub. No.: US 2019/0183807 A1
(43) Pub. Date: Jun. 20, 2019

(54) Therapeutic Agent Release System

(71) Applicant: The Johns Hopkins University, Baltimore, MD (US)

(72) Inventors: Lance M. Baird, Los Angeles, CA (US); Xiomara Calderon-Colon, Fulton, MD (US); Morgana M. Trexler, Baltimore, MD (US); Leslie H. Hamilton, Silver Spring, MD (US)

(21) Appl. No.: 16/165,187

(22) Filed: Oct. 19, 2018

Related U.S. Application Data
(60) Provisional application No. 62/608,122, filed on Dec. 20, 2017.

(51) Int. Cl. A61K 9/50; A61K 31/522; A61K 47/18; A61K 9/51

(52) U.S. Cl. CPC A61K 9/5031; A61K 31/522; A61K 9/51; A61K 9/5192; A61K 47/18

(57) ABSTRACT

A therapeutic agent release system may be provided. The therapeutic agent release system may include a plurality of polymer shells having a diameter of about 50-200 nanometers. The therapeutic agent release system may further include a bio-active therapeutic agent encapsulated by each of the polymer shells and being configured to heal an injury and increase a wound electric signal of the injury thereby increasing a healing rate of the injury. Each of the polymer shells may have a degradation profile configured to control a release of the bio-active therapeutic agent through the polymer shell to the injury over a predetermined period of time.

### CROSS-REFERENCE TO RELATED APPLICATIONS

This application claims the benefit of U.S. Provisional Application No. 62/608,122 filed on Dec. 20, 2018, the entire contents of which are hereby incorporated herein by reference.

### STATEMENT OF GOVERNMENTAL INTEREST

This invention was made with U.S. Government support under contract number W81XWH-14-1-0542 awarded by the U.S. Army. The government has certain rights in the invention.

### TECHNICAL FIELD

Exemplary embodiments of the present disclosure generally relate to a release system that is configured to enable a release of a therapeutic agent and in particular, using nanoparticles to deliver the therapeutic agent to the site of an injury.

### BACKGROUND

While eyes may account for only 0.1% of the frontal silhouette of a person, incidence of ocular injury has been shown to be high, particularly amongst military professionals and young children. Furthermore, ocular injuries are a leading cause of visual loss with some of the most common ocular injuries being to the cornea. Various drug delivery systems such as viscous ointments and polymeric hydrogels have been used in the past to treat cornea injuries. However, ointments and hydrogels suffer from the limitation of having to be reapplied and drug loss due to rapid removal of foreign materials by the cornea.

### BRIEF SUMMARY OF SOME EXAMPLES

Some example embodiments may enable the provision of a release system for a bio-active therapeutic agent. The release system of example embodiments contained herein may be configured to significantly improve the healing rate of an injury, such as an injury to the eye or the like. The release system may include a plurality of polymer shells where each polymer shell is configured to encapsulate a therapeutic or pharmaceutical agent. The therapeutic or pharmaceutical agent may be configured to be released or expelled from the polymer shell over a predetermined period of time into a wound. The release system therefore may allow for a controlled and extended release of a bio-active therapeutic agent over a predetermined time frame to the wound to aid in the healing of the injury and patient recovery time without limitations such as having to be constantly reapplied by a user.

In one example embodiment, a therapeutic agent release system may be provided. The therapeutic agent release system may include a plurality of polymer shells having a diameter of about 50-200 nanometers. The therapeutic agent release system may further include a bio-active therapeutic agent encapsulated by each of the polymer shells and being configured to heal an injury and increase a wound electric signal of the injury thereby increasing a healing rate of the injury. Each of the polymer shells may have a degradation profile configured to control a release of the bio-active therapeutic agent through the polymer shell to the injury over a predetermined period of time.

In a further example embodiment, a nanoparticle may be provided. The nanoparticle may include a hydrophobic polymer shell having a diameter of about 50-200 nanometers and a hydrophilic bio-active therapeutic agent encapsulated by the polymer shell. The hydrophilic bio-active therapeutic agent may be configured to be delivered to an area of a body and release through the polymer shell during degradation of the polymer shell. A release rate of the bio-active therapeutic agent may be based on interaction of the hydrophobic polymer shell and the hydrophilic bio-active therapeutic agent.

In an even further example embodiment, a method of encapsulating a bio-active therapeutic agent in a polymer nanoparticle may be provided. The method may include dissolving the bio-active therapeutic agent in water to form a first solution and dissolving a polymer and a first surfactant into a solvent to form a second solution. The method may further include emulsifying the first solution into the second solution to form a first emulsion and emulsifying the first emulsion into a second surfactant solution to form a second emulsion. The method may even further include filtering and purifying the second emulsion to form a nanoparticle solution containing polymer nanoparticles encapsulating the bio-active therapeutic agent, where therapeutic entrapment efficiency of the bio-active therapeutic agent or size of the nanoparticles is not affected by a molecular weight of the polymer.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates a therapeutic agent release system according to an example embodiment.

FIG. 2 illustrates a therapeutic agent release system implanted on a cornea according to an example embodiment.

FIG. 3 illustrates a nanoparticle of a therapeutic agent release system according to an example embodiment.

FIG. 4 illustrates a degradation profile of a nanoparticle according to an example embodiment.

FIG. 5 illustrates a block diagram of a method of preparing a nanoparticle according to an example embodiment.

FIGS. 6A and 6B illustrate a graphical representation of dynamic light scattering data of high molecular weight and low molecular weight nanoparticles according to an example embodiment.

FIG. 7 illustrates a graphical representation of a degradation profile of a nanoparticle according to an example embodiment.

FIG. 8 illustrates a graphical representation of a degradation profile of a nanoparticle according to a further example embodiment.

FIG. 9 illustrates a graphical representation of a degradation profile of a nanoparticle according to an even further example embodiment.

FIG. 10 illustrates a graphical representation of a kinetics model data of high molecular weight and low molecular weight nanoparticles according to an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true. U.S. Patent Application Publication No. 2016/0106888 is hereby incorporated by reference in its entirety.

Due to rapid removal of foreign materials from cornea tissues and limitations of viscous ointments and polymeric hydrogels, a release system enabling the extended release of a bio-active therapeutic agent to an injury, such as an ocular injury, during a healing process may significantly improve the healing rate of the injury. The release system and nanoparticle described herein provide for a polymeric encapsulate that is configured to slowly erode or degrade over time (e.g., a polymer nanoparticle). The system and nanoparticle may therefore allow for a controlled and extended release of the bio-active therapeutic agent over a predetermined time frame (e.g., 1-8 days) to an area of the body, such as, for example, an ocular injury or for a disease (e.g., cancer), without having to be constantly reapplied by a user.

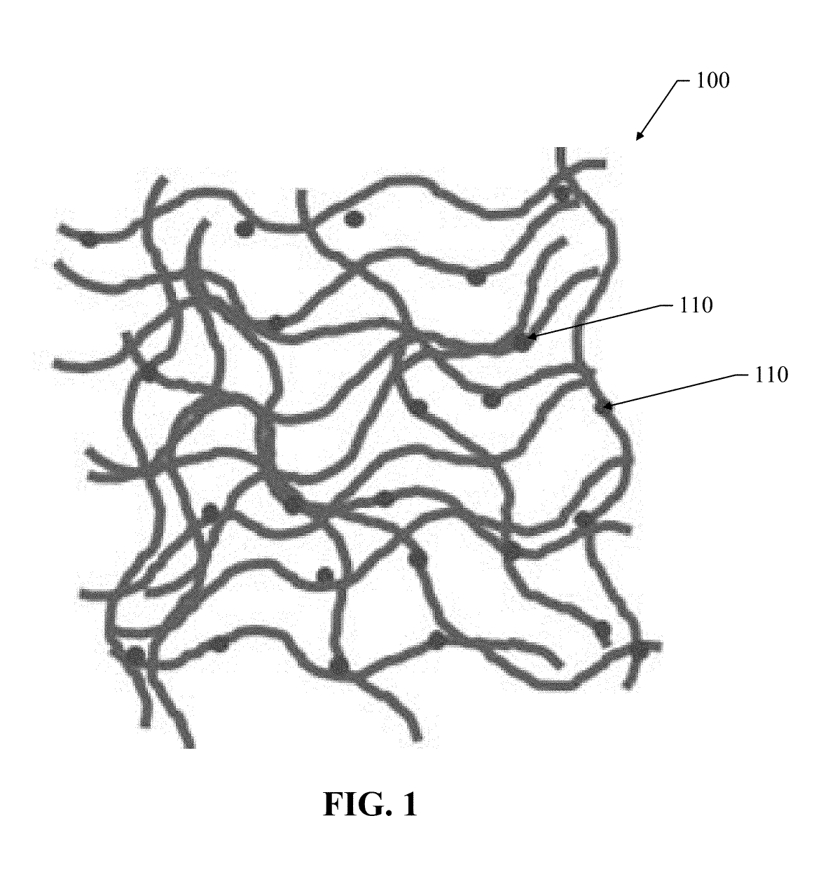

According to example embodiments contained herein and as shown in FIG. 1, a therapeutic agent release system (system) 100 may be provided. The system 100 may include a plurality of nanoparticles 110 embedded or incorporated therein. In this regard, the system 100 described herein may be an ocular implant material (e.g., collagen membrane) that is configured to be applied to an ocular injury, for example, and includes a plurality of nanoparticles 110 embedded therein.

In this regard, the system 100 described herein may be configured with a biomimetic chemistry, nanotopography, and a drug or therapeutic agent delivery functionality that may improve wound healing and in particular ocular wound healing. The ocular wound healing may be improved by 1) providing biochemical and biophysical cues for enhanced cell migration, differentiation, and proliferation and 2) delivering chemical bioelectric modulators for enhancing wound electric fields.

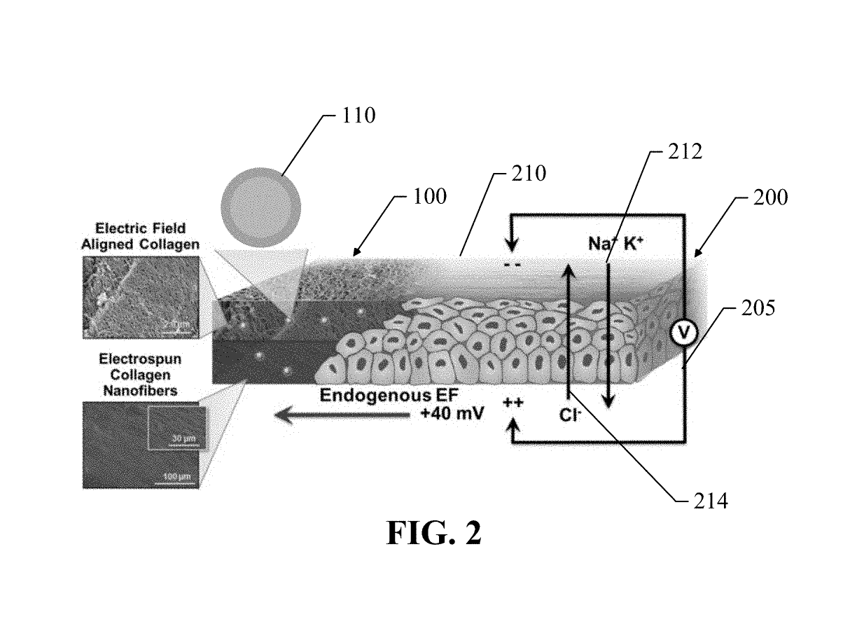

FIG. 2 illustrates an example embodiment of the release system 100 implanted on a cornea 200 of an eye. As shown in FIG. 2, a current 205 of the ocular injury (e.g., cornea injury) may initiate repair of a damaged cornea tissue 210. This current 205 may be generated by epithelial disruption of Na⁺/K⁺ ATPase pumps 212 and thus generates a lateral electric field. Wound electric fields may affect cell migration, division, proliferation, and nerve sprouting.

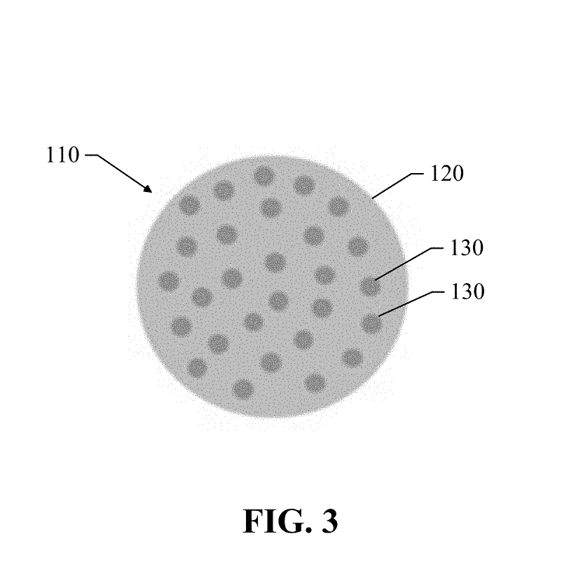

For example, in the case of the damaged cornea tissue 210, an endogenous wound electric field (EF), which arises due to active ion transport in an intact cornea epithelium surrounding the damaged cornea tissue 210, may signal cells to begin the healing process of the cornea 200. This endogenous wound EF may be a vector, which constantly points towards the wound center, and may act as a mechanism for guiding new cells into the damaged cornea tissue 210 to aid with wound healing. Certain therapeutic or pharmacological agents (e.g., aminophylline as discussed further herein), which may be encapsulated by the nanoparticle 110 as discussed below, may modulate the wound EF. When introduced into the damaged cornea tissue 210, these therapeutic or pharmacological agents may change the magnitude of the EF thereby causing a healing rate to increase or decrease depending on whether the therapeutic or pharmacological agent is configured to either stimulate or inhibit the wound EF. Accordingly, as further discussed below, the release system 100 according to example embodiments herein may include a bio-active therapeutic agent 130 (see FIG. 3) that is encapsulated in the nanoparticle 110 and is configured to increase the wound electric signal thereby increasing the healing rate to the ocular wound. In this regard, the bio-active therapeutic agent 130 may be configured to increase cAMP levels to enhance Cl⁻ pumping 214 to the damaged cornea tissue 210.

FIG. 3 illustrates an example embodiment of the nanoparticle 110 of the release system 100. The nanoparticle 110 described herein may have diameters of about 50 nanometers to 200 nanometers. Accordingly, the nanoparticle 110 may have a diameters of at least 50, 55, 60, 65, 70, 75, 80, 85, 90, 95, or 100 nanometers or at most 110, 115, 120, 125, 130, 135, 140, 145 150, 155, 160, 165, 170, 175, 180, 185, 190, 195 or 200 (e.g., about 70-100 nanometers, about 80-120 nanometers, etc.). In embodiments where the nanoparticle 110 is used for treating an ocular injury, the nanoparticle 110 may have a predefined diameter tailored such that the nanoparticle does not scatter light (e.g., about 75-125 nanometers) and thus enables efficient and effective treatment of the wound.

As shown in FIG. 3, each nanoparticle 110 may include a polymer shell 120. The polymer shell 120 may be configured to encapsulate the bio-active therapeutic agent 130 that is configured to assist in healing the ocular injury. Furthermore, the polymer shell 120 may include any polymer that is bio-compatible with the wound to be treated. In some cases, the polymer may be a hydrophobic polymer. Furthermore, the polymer may be configured to degrade in a presence of water or an aqueous environment or solution over a predetermined period of time. A polymer configured to slowly degrade may allow for an extended and controlled release of the encapsulated bio-active therapeutic agent 130 to the wound, injury, or targeted area of the body.

In some cases, the polymer may be poly(lactic-co-glycolic acid) (PLGA). PLGA is biocompatible with ocular tissues and has a molecular weight that may be tailored, as desired, in order to change a length of a release time of the bio-active therapeutic agent 130, as desired. In this regard, higher molecular weight polymers (e.g., 38,000-54,000 g/mol) exhibit a slower release rate, and lower molecular weight polymers (e.g., 7,000-17,000 g/mol) exhibit a faster release rate. The ability to alter the release rate of the bio-active therapeutic agent 130 by using different molecular weight polymers enables the release rate of the bio-active therapeutic agent 130 to be tuned by simply constructing nanoparticles composed of different molecular weight PLGA polymers. However, as discussed above, interaction between the polymer shell 120 and the bio-active therapeutic agent 130 may also influence the release rate of the bio-active therapeutic agent 130 into the wound. In some example embodiments, the specific bio-active therapeutic agent 130 used may negate any effect the molecular weight has on the release rate. In other embodiments, the polymer nanoparticles may be created using polycaprolactone or polylactic acid.

PLGA may undergo degradation through hydrolysis of its ester linkages in the presence of water or an aqueous solution thus allowing for extended release of the bio-active therapeutic agent 130. Specifically, a ratio of lactic acid to glycolic acid in PLGA may be adjusted to achieve a desired degradation rate of the polymer shell 120. In some cases, the ratio of lactic acid to glycolic acid may be about 50:50. However, in other cases, the ratio of lactic acid to glycolic acid may be about any of 10:90, 20:80, 30:70, 40:60, 60:40, 70:30, 80:20, or 90:10.

An increase in the glycolic acid component of PLGA may make the polymer shell 120 more hydrophilic. In this regard, by having a PLGA polymer with a much higher concentration of glycolic acid, the polymer shell 120 may have an increased ability to entrap the bio-active therapeutic agent 130. However, the higher concentration of glycolic acid may lead to a much more hydrophilic polymer shell 120 thus causing a faster release rate of the bio-active therapeutic agent 130. Of course, by having a PLGA polymer with a much lower concentration of glycolic acid, the polymer shell 120 may have a lower ability to entrap the bio-active therapeutic agent 130. However, the polymer shell 120 would be more hydrophobic thus causing a slower release rate of the polymer shell 120. Based on the specific components of the polymer, the entrapment efficiency of the polymer 120 may range from about 0.1-10%. (m/m) bio-active therapeutic agent entrapment efficiency. For example, when the ratio of lactic acid to glycolic acid in PLGA is about 50:50, the entrapment efficiency of the polymer shell 120 may be about 1% (m/m) which yields enough bio-active therapeutic agent 130 to provide a significant and desirable biological response in the wound.

As further shown in FIG. 3, the nanoparticle 110 may also include the bio-active therapeutic agent 130 that may be encapsulated by the polymer shell 120. The bio-active therapeutic agent 130 may be any bio-active therapeutic agent 130 that is configured to be delivered to a target area of a body to, for example, aid or assist in the healing of the wound, such as an ocular wound. As discussed above, in relation to a cornea injury, the bio-active therapeutic agent 130 may be a therapeutic agent that is configured to stimulate the wound EF to increase a healing rate of the wound. In some cases, the bio-active therapeutic agent 130 may be a water-soluble therapeutic agent. Furthermore, in cases where the polymer shell 120 is a hydrophobic polymer shell 120, the bio-active therapeutic agent 130 may be a hydrophilic therapeutic agent to enable the therapeutic agent 130 to partition out of the polymer shell 120 into the aqueous environment of the eye. Of course, it should be understood that the bio-active therapeutic agent 130 may be hydrophobic or hydrophilic and chosen based on the specific polymer shell 120 used or wound, injury, or disease to be treated.

In embodiments, where the wound is an ocular wound, the bio-active therapeutic agent 130 may be aminophylline. Aminophylline is a non-specific phosphodiesterase inhibitor that is configured to stimulate the wound EF by elevating cyclic adenosine monophosphate levels, which increase the current in the wound by increasing chloride transport (see FIG. 2). Specifically, when the wound is a cornea wound, aminophylline may increase the current in the wound by increasing chloride transport from an aqueous humor to a tear side of the cornea. Furthermore, aminophylline is a hydrophilic therapeutic agent with a log partition (log P) coefficient value of about −3.03. The log P coefficient value of aminophylline means that there is a relatively large concentration based driving force for the bio-active therapeutic agent 130 to partition out of the polymer shell 120 of the nanoparticle 110 into the eye (e.g., an aqueous environment of the cornea).

In other embodiments, the bio-active therapeutic agent 130 may be tailored to the specific injury or disease. For example, release system 100 with nanoparticles 110, may be configured to treat breast cancer, for example, and the bio-active therapeutic agent 130 could include fulverstrant.

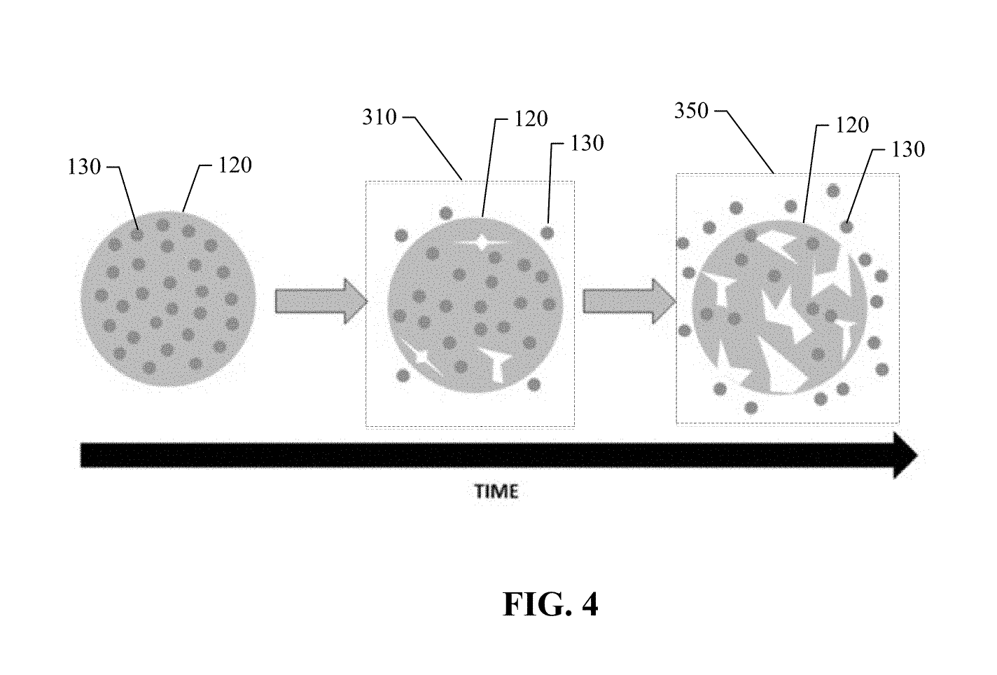

FIG. 4 illustrates an example embodiment of a degradation profile of the nanoparticle 110 of the release system 100. As mentioned above, the polymer shell 120 may be configured to degrade at a predetermined rate in order to enable the release of the bio-active therapeutic agent 130 into the wound. In this regard, the polymer shell 120 may include a degradation profile such that the bio-active therapeutic agent 130 is completely released into the wound in a predetermined period of time (e.g., 36-72 hours). In other words, the polymer shell 120 may be tailored such that the bio-active therapeutic agent is exhausted from the shell in about 36-72 hours. Accordingly, the polymer shell 120 may be configured such that the bio-active therapeutic agent is exhausted or expelled from the polymer shell 120 by 36, 40, or 48 hours or at most 50, 60, 70 or 72 hours (e.g., about 40-70 hours, about 48-72, etc.). However, in some example embodiments, the polymer shell may be configured such that the bio-active therapeutic agent may be exhausted or expelled from the polymer shell in time periods exceeding 72 hours.

The degradation profile of the polymer shell 120 may include that the bio-active therapeutic agent is released at predefined amounts in certain phases. In this regard, the degradation profile may include a bolus phase 310 and a slow release phase 350. In other words, the degradation profile may be a biphasic structure such that there is a bolus release phase 310 of the bio-active therapeutic agent 130 and a slow release phase 350 of the bio-active therapeutic agent 130. In accordance with some example embodiments, the bolus release phase 310 may be an initial burst release of the bio-active therapeutic agent 130 that happens over about 5-20 hours as a result of exposure of the nanoparticle 110 to an aqueous environment of the wound. In other words, the bolus release phase 310 may originate from osmotic pumping of the bio-active therapeutic agent 130 from a surface of the polymer shell 120 (e.g., hydrophilic bio-active therapeutic agent 130 out of the hydrophobic polymer shell 120). The slow release phase 350 may be an extended, steady release of the bio-active therapeutic agent 130 that is configured to occur after the bolus release phase 310 and happen over about 10-72 after the bolus release phase 310. The slow release phase 350 may be caused by diffusion of the bio-active therapeutic agent 130 through a matrix or pores of the polymer shell 120 or bulk hydrolysis of the polymer shell 130. Furthermore, the bolus release phase 310 may last from about 5-20 hours and the slow release phase 350 may last from about 24-70 hours. Additionally, the bolus release phase 310 may include a release of about 50-80% of the bio-active therapeutic agent 130 over about 5-20 hours and the slow release phase 310 may include a release of about 20-50% of the bio-active therapeutic agent 130 over 24-70 hours.

In other example embodiments, the degradation profile of the polymer shell 120 may be a triphasic structure such that there is an initial bolus phase, a slow release phase, and finally a fast release phase. In embodiments where the degradation profile is triphasis, the bolus release phase may be an initial burst release of the bio-active therapeutic agent 130 that is configured to happen over about 5-15 hours from the exposure of the nanoparticle 110 to the aqueous environment of the wound. The slow release phase may be an extended, steady release of the bio-active therapeutic agent that is configured to happen after the bolus release phase and over about 10-40 hours. The fast release phase may follow the slow release phase and be a burst release that happens over about 5-15 hours. Furthermore, the bolus release phase may include a release of about 10-20% of the bio-active therapeutic agent over 5-20. The slow release phase may include a release of about 10-20%, and the fast release phase may include a release of about 60-80% of the bio-active therapeutic agent over 24-70 hours.

In some example embodiments, the phases of the degradation profile may be caused by or related to the interaction of the polymer shell 120 with the bio-active therapeutic agent 130. In this regard, the bolus release phase may be caused by an initial burst of any surface bound bio-active therapeutic agents 130 out of the polymer shell 120. In other words, the bolus release phase may originate from osmotic pumping of the bio-active therapeutic agent (e.g., hydrophilic bio-active therapeutic agent). The slow release phase may be caused by diffusion of the bio-active therapeutic agent 130 through a matrix or pores of the polymer shell 120. In embodiments that include a fast release phase, the fast release phase may be caused by bulk hydrolysis of the polymer shell 120.

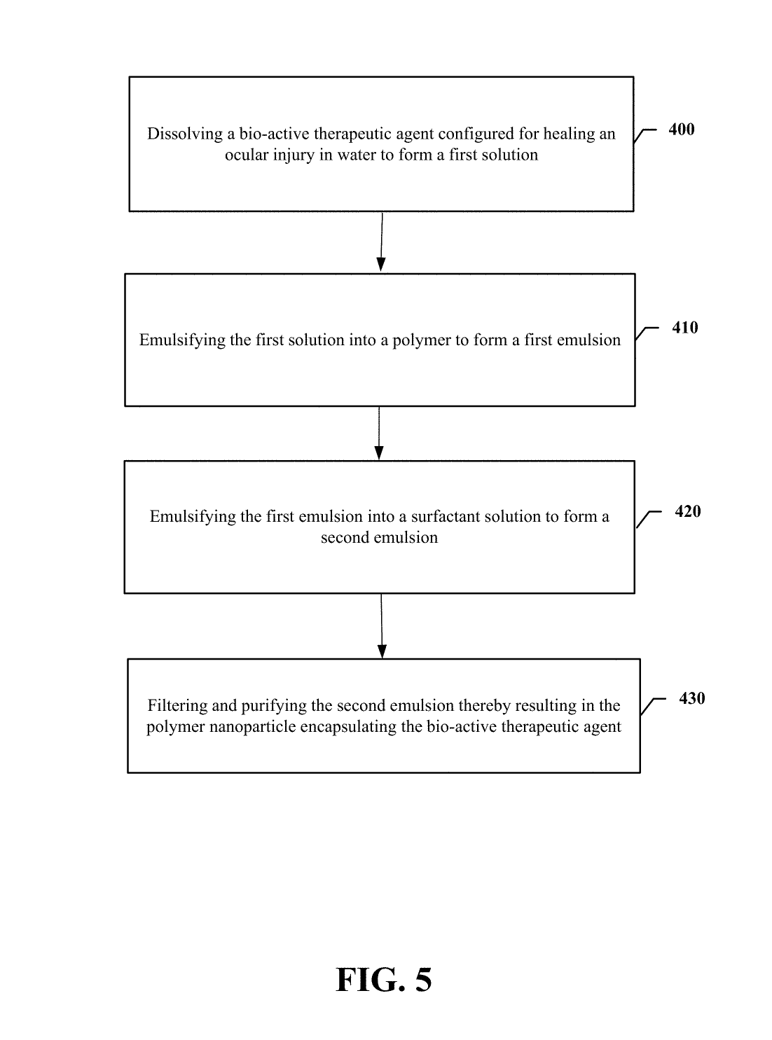

FIG. 5 illustrates a block diagram of a method of preparing the nanoparticle 110 described herein. The method may include, at step 400, dissolving a bio-active therapeutic agent 130, such as aminophylline, in water to form a first aqueous solution. The method may even further include, at step 410, emulsifying the first aqueous solution into a polymer solution to form a first emulsion. The emulsifying of the solutions may be done via sonication or the like. In some cases, the polymer solution may include a polymer such as PLGA. In other cases, the polymer solution may include the polymer and a solvent. The solvent may include ethyl acetate or the like. In even further cases, the polymer solution may include the polymer, the solvent, and a surfactant. The surfactant may be a non-ionic surfactant such as Pluronic F-68. In this regard, the polymer solution may include a combination of the polymer and any of the solvent and the surfactant. Furthermore, the emulsification of the first aqueous solution with the polymer solution may result in a water in oil emulsion.

The method may further include, at step 420, emulsifying, via sonication or the like, the first emulsion in a surfactant solution to form a second emulsion. The surfactant solution may include a non-ionic surfactant such as Pluronic F-68. Furthermore, the emulsification of the first emulsion with the surfactant solution may result in a water in oil in water emulsion. The method may even further include, at step 430, filtering and purifying the second emulsion thereby resulting in the nanoparticle 110 that includes the polymer 120 encapsulating the bio-active therapeutic agent 130. In some cases, step 430 includes removing the solvent to precipitate the polymer and form the polymer shell 120. In this regard, a vacuum may be applied to the second emulsion and the solvent may be allowed to evaporate. The vacuumed emulsion may then be purified using a filtration system to yield a nanoparticle solution. After purification, the nanoparticle solution may be dried in order to precipitate the polymer. Finally, the nanoparticle solution may be further purified, via dialysis or the like, to yield the nanoparticle 110. It should be understood that the method used for producing the nanoparticle 110 is not limited to the method discussed above but may include any of a single or double emulsion solvent evaporation, nanoprecipitation, salting-out, membrane emulsification, microfluidics, or flow focusing.

In an embodiment, poly(lactic-co-glycolic acid) nanoparticles contain the therapeutic aminophylline. The nanoparticles have an average diameter of 125 nanometers, and are composed of a poly(lactic-co-glycolic acid) polymer shell with an aqueous core containing 6% (m/m) aminophylline, for example. The use of these particles includes the extended release of aminophylline in a collagen cornea implant material, so as to expedite the healing rate of a corneal injury.

In an embodiment, the nanoparticles are synthesized through a double emulsion and solvent evaporation process. This involves first dissolving the aminophylline into water. Then, emulsifying this aqueous solution into an organic solvent composed of ethyl acetate, poly(lactic-co-glycolic acid), and a surfactant. The water in oil emulsion is then emulsified into a water/surfactant solution to create a water in oil in water (w/o/w) emulsion. Once the w/o/w emulsion is formed, the ethyl acetate solvent is removed using a rotary evaporator to precipitate the PLGA and form the nanoparticle shell. The nanoparticle solution was purified via dialysis and lyophilized to yield a free flowing white powder.

Accordingly, some example embodiments may enable the provision of a release system 100 for a bio-active therapeutic agent 130. The release system 100 may be configured to significantly improve the healing rate of an injury, such as an injury to the eye or the like. In this regard, the release system 100 may include a plurality of nanoparticles 110. Each nanoparticle 110 may include a polymer shell 120 that is configured to encapsulate a therapeutic or pharmaceutical agent 130. The therapeutic or pharmaceutical agent 130 may be configured to be released or expelled from the polymer shell 120 over a predetermined period of time into a wound.

The following example is provided to enable one of skilled in the art to practice the invention and is merely illustrative and in no way should be construed as being limiting. In this regard, the example should not be read as limiting the scope of the present disclosure.

Reagent:

Ethyl acetate (CAS [141-78-6]); pluronic F68 (CAS [9003-11-6]); aminophylline (CAS [317-34-0]); poly(lactic-co-glycolic acid) 50:50 MW=38,000-54,000 (CAS [26780-50-7]; acid terminated, poly(lactic-co-glycolic acid) 50:50 MW=7,000-17,000 (CAS [26780-50-7]; acid terminated, premixed phosphate buffered saline (PBS) buffer (sigma 11666789001); sucrose (CAS [57-50-1]); ammonium formate (CAS [540-69-2]); formic acid (CAS [64-18-6]); Spectrum Labs KR2i tangential flow filtration system with a mPES MidiKros® 100 KDa MWCO filter module; and a Pur-A-Lyzer dialysis kit with a molecular weight cut-off (MWCO) of 7-17 kDA were used.

Nanoparticle Preparation:

For a 650 mg batch of nanoparticles, 1 g of aminophylline was dissolved into 10 mL of DI water. A solution composed of 0.9 g of the PLGA polymer and 0.9 g of Pluronic F68 were dissolved into 30 mL of ethyl acetate. The aminophylline solution was transferred into the ethyl acetate/PLGA/Pluronic F68 solution and sonicated at 20% maximum amplitude for 90 seconds using a Misonix horn sonicator model S-4000. The sonicated solution was kept cool with an ice/water bath to reduce heating and solvent evaporation. This water-in-oil emulsion was placed into 100 mL of 3% (w/v) Pluronic F68 dissolved in DI water and sonicated for 90 seconds at 90% maximum amplitude. This solution was also placed over an ice/water bath during sonication to reduce heating. The resulting water/oil/water (w/o/w) emulsion was then placed into a rotary evaporator where vacuum was applied and the solvent was allowed to evaporate off for one hour.

The rotovaped solution was purified using a Spectrum Labs KR2i tangential flow filtration system equipped with a 100 MWCO hollow fiber dialysis tube (Midikros D02-E100-05-N). For the purification, a continuous dial filtration process was utilized in which 1 L of DI water at a flow rate of 150 mL/min and a transmembrane pressure of 10 psi were used to remove any low molecular weight impurities and excess surfactant. The tangential flow filtration process took approximately one hour to complete and yielded a 100 mL solution containing 6.5 mg/mL of PLGA nanoparticles. After purification, the nanoparticle solution was then mixed with 5 g of sucrose and lyophilized using a Labconco Freezone Triad freeze drier. The lyophilized samples were stored in a 4° C. refrigerator to inhibit the hydrolysis of the polymer.

Particle Size and Polydispersity:

For particle size determination, dynamic light scattering (DLS) data of the particles were obtained using a Malvern Zetasizer Nano S dynamic light scattering system. NIST traceable polystyrene latex standards were used to validate the calibration of the instrument before each measurement.

Therapeutic Release:

For construction of the in vitro release profiles, a 900 mg aliquot of the lyophilized PLGA nanoparticle sample was suspended in 4.5 mL of a 0.1M PBS buffer. This buffer/nanoparticle suspension was placed in a Pur-A-Lyzer Mini Dialysis Device, and the cell was submerged into 45 mL of the PBS buffer. The devices were kept in an isotemp incubator at a temperature of 34° C. (temperature similar to that of the human cornea) and slowly stirred. At each time point, the supernatant liquid was collected and another 45 mL of fresh PBS buffer solution (34° C.) was added to maintain sink conditions for the entirety of the experiment. There were 10 data points taken for each PLGA nanoparticle sample at time intervals of: 0.5, 2, 4, 8, 12, 24, and 48 hours. These data were then used to construct aminophylline release profile curves and perform the kinetics analysis.

Liquid chromatography-mass spectrometry (LC-MS) was used as the analytical technique for quantifying the amount of aminophylline in each one of the release study time points. In our LC-MS method, an AB Sciex API-2000 LC-MS instrument equipped with a Waters Xbridge (C19, 3.5 μm, 2.1×150 mm, SN: 01803611014045) was used. The mobile phase was composed of 10 mM ammonium formate and 0.1% formic acid in DI water. An injection volume of 10 μl, column temperature of 75° C., and run time of 20 minutes were used for sample analysis. For analysis of aminophylline, the LC-MS method had an accuracy of ±3%, precision of 4%, LOQ of 0.039 ppm, and a linear range of 0.078-2.5 ppm.

Synthesis:

PLGA nanoparticles that had 1% (m/m) aminophylline entrapment efficiency were produced, which is on the upper end of the spectrum for entrapment efficiency using this technique. The entrapment efficiency was calculated based on mass of aminophylline in particles divided by a total mass of particle. Two sets of nanoparticles using the double emulsion solvent extraction (DESE) method were successfully synthesized. PLGA with a 50:50 ratio of lactic to glycolic acid was used as the polymer shell material for the production of these nanoparticles, all of the synthesis variables were held constant except for the molecular weight of the PLGA polymer used to produce the polymer shell. This was done to determine what influence the PLGA molecular weight may have on the therapeutic delivery properties of the particles.

Nanoparticle Size and Therapeutic Entrapment:

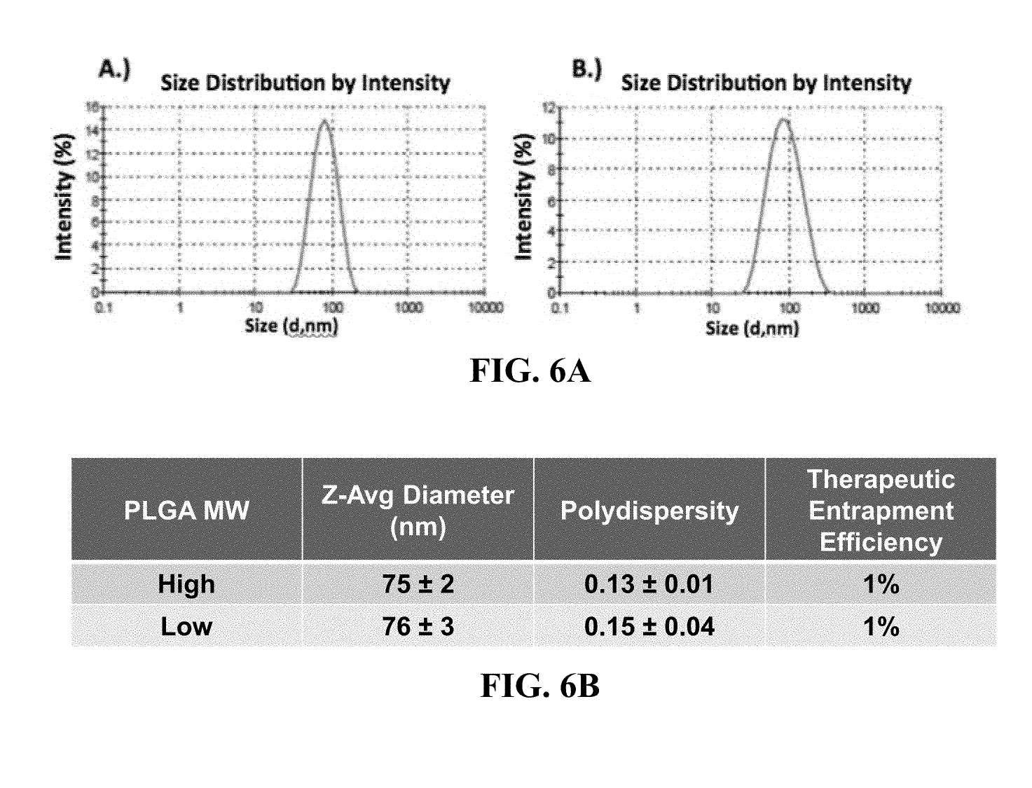

FIGS. 6A and 6B illustrate the dynamic light scattering (DLS) data for the two PLGA molecular weights used to produce the nanoparticles are described. As shown in FIG. 6B the particle size for the low and high molecular weight was 76±3 nm and 75±2 nm, respectively. The DLS data showed monodisperse PLGA nanoparticles; data in FIG. 6B was averaged for three batches of nanoparticles (see FIG. 6A). Moreover, PLGA molecular weight was not shown to affect the therapeutic entrapment efficiency or the size of the nanoparticles. This observation runs contrary to data known in the art, in which it has been shown that therapeutic entrapment efficiency is directly proportional to the molecular weight of the polymer used to synthesize the particles. This observation is in part due to entrapping a hydrophilic therapeutic molecule into the PLGA polymer. Due to the hydrophilic therapeutic molecule wanting to diffuse out of the PLGA shell, which is somewhat hydrophobic, and flow into the aqueous reservoir, a strong partitioning force is created and overcomes the effect of PLGA molecular weight on the release kinetics of the therapeutic molecule. In other words, the hydrophilic molecule (e.g., aminophylline) partitioning force was greater than the effect of increasing the PLGA molecular weight. It should be understood, however, that if a hydrophobic drug were encapsulated into the PLGA nanoparticles, the molecular weight would have an effect on the therapeutic release kinetics.

Accordingly, unlike examples in the current state of the art, examples herein involve hydrophilic therapeutic aminophylline entrapped into PLGA nanoparticles. The log partition (log P) coefficient value of aminophylline is −3.03, which means that there is a relatively large concentration based driving force for the therapeutic to partition out of the highly concentrated aqueous core of the nanoparticle and into the continuous aqueous phase during the nanoparticle synthesis. In this case, the concentration driven diffusion process for aminophylline to partition into the aqueous continuous phase masked any effect that the polymer molecular weight had on entrapment efficiency. It is for this reason that the entrapment efficiency was roughly identical for the low and high molecular weight PLGA nanoparticles.

Lyophilization Effect:

To inhibit the hydrolysis of synthesized PLGA nanoparticles, these materials were lyophilized. Initially, only agglomerated solids were formed after lyophilization of nanoparticles made from both the low and high molecular weight PLGA polymers. It was thought that this agglomeration was due to the solvent used during the nanoparticle synthesis not being fully removed during the evaporation synthesis step. Even after extended solvent evaporation times were used for synthesizing the nanoparticles, the lyophilized nanoparticles still exhibited agglomeration and were not able to be easily dispersed in DI water.

The agglomeration of the low molecular weight PLGA nanoparticles occurred due to mechanical stresses created during the lyophilization process. As the nanoparticle concentration increased during the sublimation of water, the interaction between these particles led them to aggregate and fuse together. Additional stress was incurred on the particles due to the crystallization of ice. The ice crystallization mechanism is thought to exhibit a mechanical stress on the nanoparticles leading to their destabilization and eventual fusion. To protect the nanoparticles against the formation of these mechanical stresses during lyophilization, cryoprotectants such as trehalose, glucose, sucrose, and mannitol can be used. These sugars vitrify at a very specific temperature, which immobilizes the nanoparticles into a glassy matrix preventing their aggregation and protecting them against the mechanical stress of ice crystal formation.

Out of all the cryoprotectants discussed above, sucrose was chosen because of its affordability. With the addition of 5 g of sucrose to a 100 mL aqueous nanoparticle solution, the agglomeration effects previously seen during lyophilization were completely abolished. Both high and low molecular weight PLGA nanoparticles lyophilized with the addition of sucrose yielded free-flowing powders that were easily dispersed into DI water.

Release Profiles:

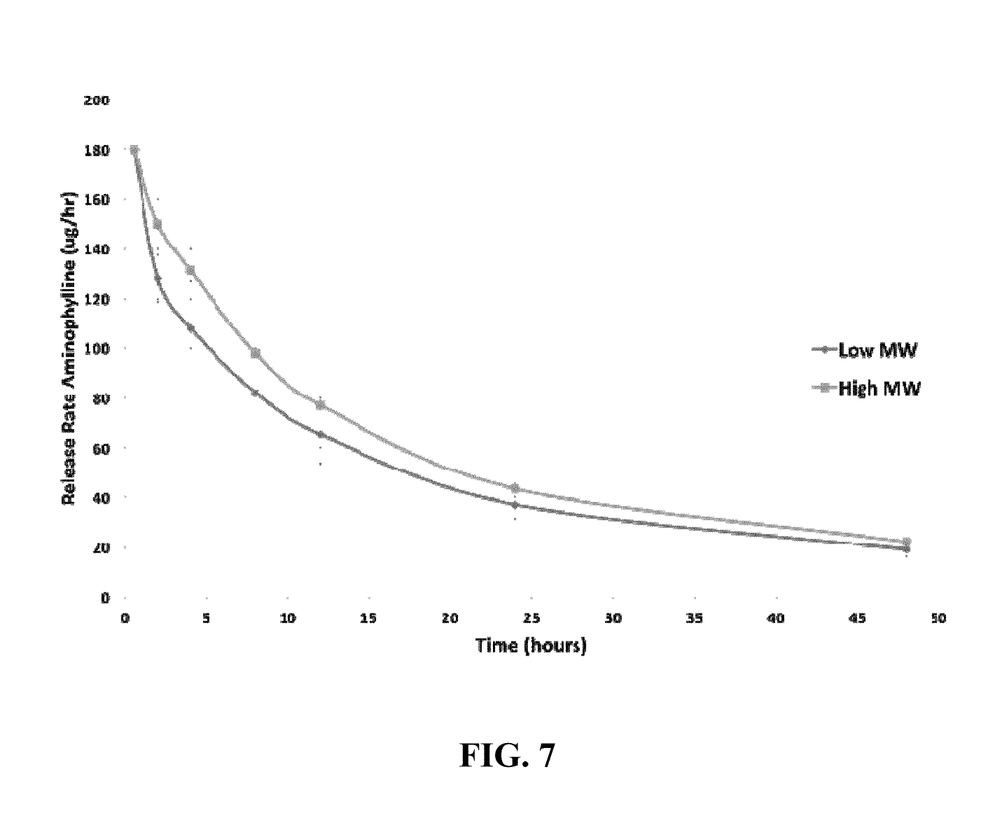

FIGS. 7-9 illustrate example release profiles of the nanoparticle according to example embodiments herein. The release profile curves were generated using a dynamic dialysis technique to study the release of aminophylline from the PLGA nanoparticles. There are several other methods (based on centrifugation techniques) for monitoring the release of therapeutics from nanoparticles. These techniques were not chosen because they involve lengthy centrifugation methods which can lead to sample loss. The dynamic dialysis method was chosen because the additional step of separating the nanoparticles from the free drug at various monitoring times during the analysis is completely removed, thereby making the sampling technique much more robust and easier on the operator. In addition, the external pressure applied for separation in other methods can disturb the equilibrium and allow for incomplete separation and significant measurement errors. Even though the dynamic dialysis method is a much simpler technique, it is important to keep in mind that the apparent release rate is the net result of therapeutic transport across two barriers in series, and the release kinetics may not reflect the rate of therapeutic release from the nanoparticles alone.

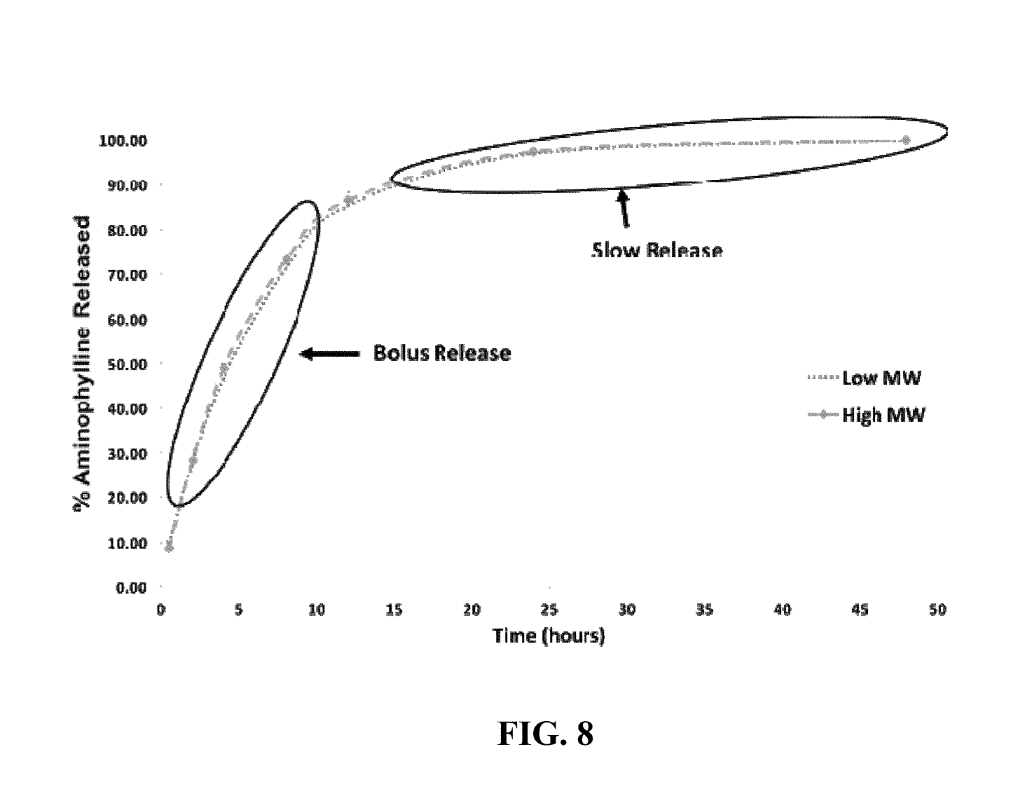

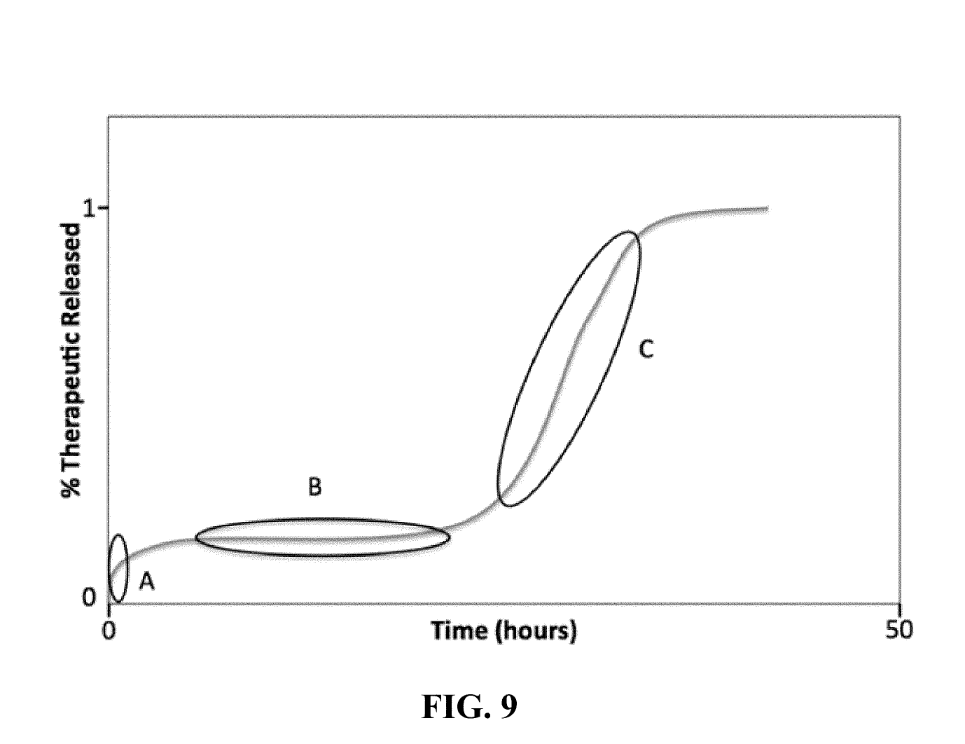

In the generated aminophylline release curves illustrated in FIGS. 7 and 8, it is evident that the two sets of nanoparticles have identical release profiles. Each data point in the release profiles represents the mean of three measurements. As can be seen in FIG. 8, both samples exhausted their store of aminophylline in approximately 48 hours. The release profiles for high and low molecular weight PLGA nanoparticles in FIG. 8 exhibited a biphasic structure, in which a bolus release was followed by a slow release phase. The bolus release originated from osmotic pumping of the relatively low molecular weight aminophylline, whereas the slow release phase was due to diffusion of the therapeutic through the PLGA matrix and inherent pores. Therapeutic release from PLGA nanoparticles generally are triphasic, with a bolus release phase occurring first, then slow drug diffusion, and finally bulk erosion of the polymer (see FIG. 9). Since no bulk erosion phase was observed for either the high or low molecular weight nanoparticles, the hydrolysis of the PLGA shell was not an important variable in the therapeutic release kinetics for the specific example described herein. However, the release profiles may be tailored based on the polymer used to create the shell or the therapeutic agent or molecule encapsulated therein.

The presence of identical biphasic release profiles for the low and high molecular weight PLGA nanoparticles may be due to the hydrophilic nature of aminophylline and the propensity for the nanoparticle polymer shell to swell in an aqueous environment. Since the entrapped therapeutic has such a low log P value, it is safe to assume that there is a strong concentration dependent force driving the diffusion of the therapeutic into the continuous aqueous phase. In addition, as the PLGA polymer begins to swell, this will cause the pores in the nanoparticle shell to dilate, thus causing an increased release of aminophylline. These two factors in combination help to explain why the differences in molecular weight, which is directly related to polymer hydrolysis rate, had no significant effect on the release rate of the therapeutic.

Release Kinetics Evaluation:

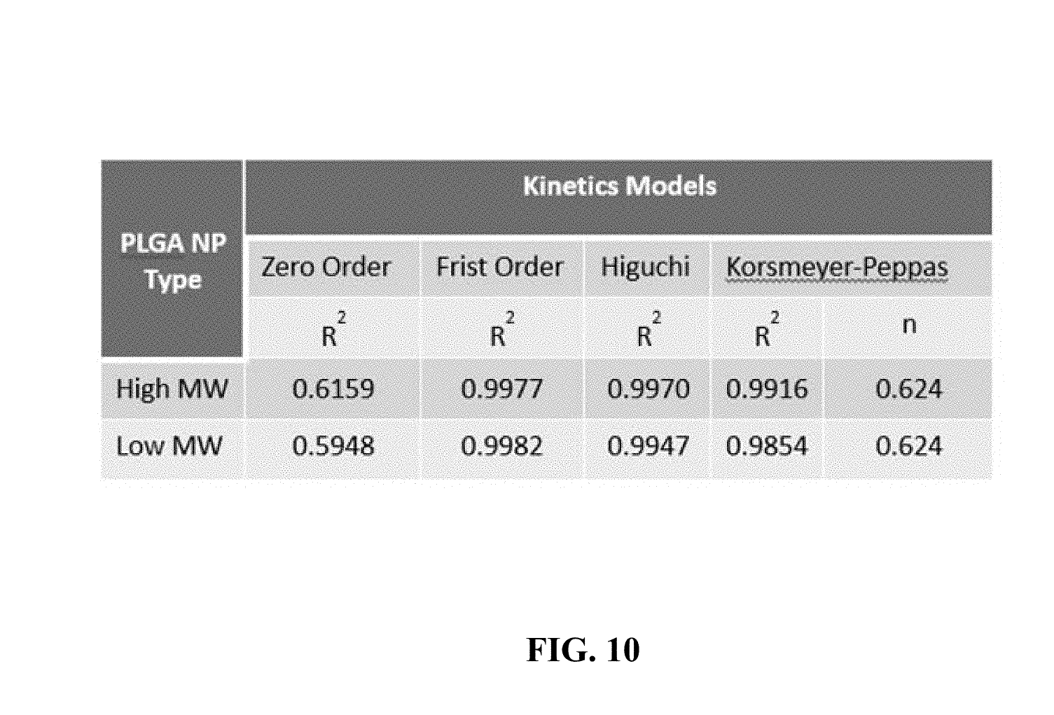

To further explain the similar release profiles exhibited by both the low and high MW PLGA nanoparticles, a kinetics analysis was undertaken. FIG. 10 illustrates the kinetic analysis in accordance with an example embodiment. The release profile data of the nanoparticles were fitted to several models: zero order, first order, Higuchi, and Korsemeyer-Peppas. The equation describing Zero Order kinetics is Qt=Q0+Kt, and that describing First Order kinetics is log Qt=Log Q0−Kt/2.303. In these equations Qt is the cumulative drug release at time t, Q0 is initial amount of drug, K is the respective release constant, and t is the time. Evaluating the fits to these models revealed that the release of aminophylline from both the low and high MW PLGA nanoparticles exhibited first order release profiles. Further evaluation of the release profiles using the Higuchi equation, Qt/Q0=Kt1/2, and the Korsmeyer-Peppas equation, Qt/Q0=Kmtn, provided further insight into the exact mechanisms by which the therapeutic was released. For the Korsmeyer-Peppas equation n is the diffusion release exponent. The coefficient of determination (R2) values were used to determine the model that best fit the nanoparticle release rate data. As shown in FIG. 10, the release profile data for both PLGA types fit very well with the first order kinetic model (R2 values of 0.9977 and 0.9982 respectively for the high and low MW PLGA). This correlates well with the observed release profiles, and signifies that the diffusion process of aminophylline was concentration-dependent for both molecular weight samples.

In order to elucidate the diffusion process by which the aminophylline was released from the nanoparticles, the data was fitted to both Higuchi and Korsmeyer-Peppas models. The release profile data correlated very well (R2 or 0.9970 and 0.9947 for high and low MW nanoparticles) to the Higuchi model, which denoted that the release rate was a diffusion controlled process resulting from the therapeutic agent being released from a matrix material. Fitting the therapeutic release data to the Korsmeyer-Peppas model helped to further describe exactly what type of diffusion process (Fickian or anomalous/non-Fickian) the therapeutic followed. With a diffusion release exponent calculated to be 0.624 for both MW PLGA nanoparticles, the diffusion process was determined to be anomalous/non-Fickian. With non-Fickian diffusion process, there is a direct relationship between cumulative therapeutic release and time. Since anomalous/non-Fickian diffusion relates to drug release from a combination of diffusion and pore swelling, this model fit correlated well to the experimental data.

Thus, in accordance with example embodiments herein, a therapeutic agent release system may be provided. The therapeutic agent release system may include a plurality of polymer shells having a diameter of about 50-200 nanometers. The therapeutic agent release system may further include a bio-active therapeutic agent encapsulated by each of the polymer shells and being configured to heal an injury and increase a wound electric signal of the injury thereby increasing a healing rate of the injury. Each of the polymer shells may have a degradation profile configured to control a release of the bio-active therapeutic agent through the polymer shell to the injury over a predetermined period of time.

In some embodiments, the features described above may be augmented or modified, or additional features may be added. These augmentations, modifications, and additions may be optional and may be provided in any combination. Thus, although some example modifications, augmentations and additions are listed below, it should be appreciated that any of the modifications, augmentations and additions could be implemented individually or in combination with one or more, or even all of the other modifications, augmentations and additions that are listed. As such, for example, the bio-active therapeutic agent may be a water-soluble bio-active therapeutic agent. Additionally or alternatively, the bio-active therapeutic agent may be a hydrophilic bio-active therapeutic agent and each of the polymer shells may be a hydrophobic polymer shell. Additionally or alternatively, the degradation profile may include a bolus release phase and a slow release phase. The bolus release phase may include about 5-20 hours and the slow release phase comprises about 24-70 hours. Additionally or alternatively, the bio-active therapeutic agent may be a hydrophilic bio-active therapeutic agent and each of the polymer shells is a hydrophobic polymer shell, and the degradation profile may include an initial bolus release phase and then a slow release phase, the initial bolus release phase originating from osmotic pumping of the hydrophilic bio-active therapeutic agent. Additionally or alternatively, in order to increase a wound electric signal, the bio-active therapeutic agent is configured to increase cAMP levels thereby enhancing Cl⁻ pumping to the injury. Additionally or alternatively, each of the polymer shells may be a poly(lactic-co-glycolic acid) (PLGA) shell. Additionally or alternatively, the bio-active therapeutic agent may be aminophylline. Additionally or alternatively, the injury may be an ocular injury and the bio-active therapeutic agent may be aminophylline. Additionally or alternatively, the hydrophilic bio-active therapeutic agent may include a log partition coefficient value of about −3.0 and may force the release of the bio-active therapeutic agent through the polymer shell in response to being exposed to an aqueous environment. Additionally or alternatively, the release rate of the bio-active therapeutic agent may be further based on dilation of pores of the hydrophobic polymer shell in response to being exposed to the aqueous environment. Additionally or alternatively, in response to exposure to an aqueous environment of an eye, the release rate of the hydrophilic bio-active therapeutic agent may be an initial bolus release originating from osmotic pumping of the hydrophilic bio-active therapeutic agent and then a slow release. Additionally or alternatively, the method of encapsulating the bio-active therapeutic agent in a polymer nanoparticle may include dissolving a cryoprotectant into the nanoparticle solution to form a third solution and lyophilizing the third solution.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

That which is claimed:

1. A therapeutic agent release system comprising:
a plurality of polymer shells having a diameter of about 50-200 nanometers; and
a bio-active therapeutic agent encapsulated by each of the polymer shells and being configured to heal an injury and increase a wound electric signal of the injury thereby increasing a healing rate of the injury,
wherein each of the polymer shells has a degradation profile configured to control a release of the bio-active therapeutic agent through the polymer shell to the injury over a predetermined period of time.

2. The therapeutic agent release system of claim 1, wherein the bio-active therapeutic agent is a water-soluble bio-active therapeutic agent.

3. The therapeutic agent release system of claim 1, wherein the bio-active therapeutic agent is a hydrophilic bio-active therapeutic agent and each of the polymer shells is a hydrophobic polymer shell.

4. The therapeutic agent release system of claim 1, wherein the degradation profile comprises a bolus release phase and a slow release phase.

5. The therapeutic agent release system of claim 4, wherein the bolus release phase comprises about 5-20 hours and the slow release phase comprises about 24-70 hours.

6. The therapeutic agent release system of claim 1, wherein the bio-active therapeutic agent is a hydrophilic bio-active therapeutic agent and each of the polymer shells is a hydrophobic polymer shell, and wherein the degradation profile comprises an initial bolus release phase and then a slow release phase, the initial bolus release phase originating from osmotic pumping of the hydrophilic bio-active therapeutic agent.

7. The therapeutic agent release system of claim 1, wherein in order to increase a wound electric signal, the bio-active therapeutic agent is configured to increase cAMP levels thereby enhancing Cl⁻ pumping to the injury.

8. The therapeutic agent release system of claim 1, wherein each of the polymer shells is a poly(lactic-co-glycolic acid) (PLGA) shell.

9. The therapeutic agent release system of claim 1, wherein the injury is an ocular injury and the bio-active therapeutic agent is aminophylline.

10. A nanoparticle comprising:
a hydrophobic polymer shell having a diameter of about 50-200 nanometers; and
a hydrophilic bio-active therapeutic agent encapsulated by the polymer shell,
wherein the hydrophilic bio-active therapeutic agent is configured to be delivered to an area of a body and release through the polymer shell during degradation of the polymer shell, and
wherein a release rate of the bio-active therapeutic agent is based on interaction of the hydrophobic polymer shell and the hydrophilic bio-active therapeutic agent.

11. The nanoparticle of claim 10, wherein the hydrophilic bio-active therapeutic agent comprises a log partition coefficient value of about −3.0 and forces the release of the bio-active therapeutic agent through the polymer shell in response to being exposed to an aqueous environment.

12. The nanoparticle of claim 11, wherein the release rate of the bio-active therapeutic agent is further based on dilation of pores of the hydrophobic polymer shell in response to being exposed to the aqueous environment.

13. The nanoparticle of claim 10, wherein in response to exposure to an aqueous environment of an eye, the release rate of the hydrophilic bio-active therapeutic agent is an initial bolus release originating from osmotic pumping of the hydrophilic bio-active therapeutic agent and then a slow release.

14. The nanoparticle of claim 10, where the hydrophilic bio-active therapeutic agent is configured to heal an ocular injury by increasing a wound electric signal of the ocular injury thereby increasing a healing rate of the ocular injury.

15. The nanoparticle of claim 10, wherein the hydrophobic polymer shell is a poly(lactic-co-glycolic acid) (PLGA) shell.

16. The nanoparticle of claim 15, wherein the hydrophilic bio-active therapeutic agent is aminophylline.

17. A method of encapsulating a bio-active therapeutic agent in a polymer nanoparticle, the method comprising:
dissolving the bio-active therapeutic agent in water to form a first solution;
dissolving a polymer and a first surfactant into a solvent to form a second solution;
emulsifying the first solution into the second solution to form a first emulsion;
emulsifying the first emulsion into a second surfactant solution to form a second emulsion; and
filtering and purifying the second emulsion to form a nanoparticle solution containing polymer nanoparticles encapsulating the bio-active therapeutic agent, wherein therapeutic entrapment efficiency of the bio-active therapeutic agent or size of the nanoparticles is not affected by a molecular weight of the polymer.

18. The method of claim 17, wherein the polymer is poly(lactic-co-glycolic acid) (PLGA).

19. The method of claim 17, wherein the bio-active therapeutic agent is aminophylline.

20. The method of claim 17 further comprising:
dissolving a cryoprotectant into the nanoparticle solution to form a third solution; and
lyophilizing the third solution.
